(19) United States
(12) Patent Application Publication
(10) Pub. No.: US 2009/0140133 A1
(43) Pub. Date: Jun. 4, 2009

(54) Pipeline pig and method for irradiation of bacteria in a pipeline

(75) Inventor: Laurence Abney, Sandnes (NO)

Correspondence Address: JOHN W. WUSTENBERG, P.O. BOX 1431, DUNCAN, OK, 73536, US

(73) Assignee: Halliburton Energy Services, Inc., Houston, TX (US)

(21) Appl. No.: 11/947,660

(22) Filed: Nov. 29, 2007

(51) Int. Cl. G01N 23/00

(52) U.S. Cl. 250/253; 250/493.1

(57) ABSTRACT

A pipeline pig includes a fluid driven electrical power generator and an ultraviolet radiation source powered by the electrical power generator. Radiation is directed away from the pig to irradiate the inner surface of a pipeline when the pig is driven through the pipeline by fluid and thereby disinfects the inner surface of the pipeline.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### FIELD OF THE INVENTION

The present invention relates generally to apparatus using radiation, such as ultraviolet light, to disinfect pipelines, and more particularly to a pipeline pig which irradiates the inner surface of a pipeline to destroy or inactivate biological material on inner surfaces of the pipeline.

### BACKGROUND

Pipelines and other flow conduits are used in a variety of industries to transport a variety of products. In the hydrocarbon industry, pipelines and other flow conduits are used for transporting unprocessed hydrocarbon fluids and processed products such as stabilized crude oil and/or gas. Additionally, in the hydrocarbon industry, pipelines are used to transport refined and processed products such as gasoline, diesel, aviation fuel and dehydrated gas. During the fabrication and installation of pipelines, lengths of pipe are welded together to form a continuous pipeline. Once the pipeline has been fabricated and installed, it must be tested to prove that the integrity of the pipeline is such that when filled with product the product will not leak into the environment outside of the pipeline. Part of the testing process typically involves filling a pipeline with water so that it can be subjected to a hydrostatic test. During the filling process, chemicals have been typically injected into the fill and test water to kill any bacteria. These chemicals are known as biocides. The biocides not only kill the bacteria in the water, but they also kill any bacteria that may be present on the internal walls of the pipeline. After testing, the water must be removed from the pipeline and properly disposed of. The biocides in the test water destroy biological matter and therefore the water usually cannot be released into the environment until the biocides are removed or deactivated.

To avoid the problem created by the presence of biocides in the test water, alternative (non-chemical) systems are available to kill or inactivate bacteria in the water as it is injected into the pipeline. However, these systems do not affect any bacteria or biological material that may be present on the inner surface of the pipeline.

Biological material, e.g. bacteria, are known to react with hydrocarbon products, e.g. oil or gas, or materials carried in such products, e.g. sulfur or sulfates, to produce corrosive materials which can damage pipelines. It is therefore desirable to both use disinfected test fluids and to remove or deactivate any biological material on the inner surface of pipelines regardless of the origins of such materials.

### SUMMARY OF THE INVENTION

A pipeline pig includes a fluid driven power generator and a radiation source powered by the power generator. Radiation is directed away from the pig to irradiate the inner surface of a pipeline when the pig is driven through the pipeline by a liquid or gaseous fluid and thereby disinfects the inner surface of the pipeline.

In an embodiment, the radiation source emits ultraviolet radiation.

### BRIEF DESCRIPTION OF THE DRAWINGS

For a more detailed description of the present invention, reference will now be made to the accompanying drawings, wherein:

FIG. 1 is a schematic diagram of a typical pipeline pre-commissioning operation in which embodiments of the present invention may be used.

FIG. 2 is a partially schematic diagram of an embodiment of an apparatus employing an irradiation system to disinfect the inner surfaces of a pipeline.

### NOTATION AND NOMENCLATURE

Certain terms are used throughout the following description and claims to refer to particular assembly components. This document does not intend to distinguish between components that differ in name, but not function. In the following discussion and in the claims, the terms “including” and “comprising” are used in an open-ended fashion, and thus should be interpreted to mean “including, but not limited to . . . .”

As used herein, each of the terms “disinfect”, “kill, and “inactivate” means to render biologically inert. This includes killing the biological material or destroying the ability of the material to reproduce or any other mechanism which terminates the ability of the material to produce corrosive materials, at least after a period of time.

As used herein, the term “pipeline” includes any line in which fluid is moved, including any onshore or offshore flow system, such as mainline systems, risers, flow lines used to transport untreated fluid between a wellhead and a processing facility, and flow lines used to transport hydrocarbon products. It should be understood that the use of the term “pipeline” is not necessarily limited to hydrocarbon pipelines unless otherwise denoted or required by a specific embodiment.

In the drawings, the arrows indicate the direction of fluid flow through the system in a sequential operation.

### DETAILED DESCRIPTION

Various embodiments of apparatus and methods for treating a pipeline, by way of non-limiting example a pipeline for use in hydrocarbon industry applications, will now be described with reference to the accompanying drawings, wherein like reference numerals are used for like features throughout the several views. There are shown in the drawings, and herein will be described in detail, specific embodiments of irradiation systems and methods of using such systems to disinfect pipelines, with the understanding that this disclosure is representative only and is not intended to limit the invention to those embodiments illustrated and described herein. The embodiments of irradiation systems disclosed herein may be utilized in any type of industrial process, including without limitation any hydrocarbon industry application, operation, or process where it is desired to disinfect pipelines including; well servicing operations, upstream exploration and production applications, and downstream refining, processing, storage and transportation applications. It is to be fully recognized that the different teachings of the embodiments disclosed herein may be employed separately or in any suitable combination to produce desired results.

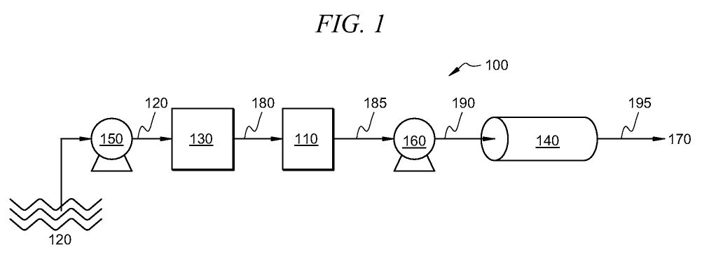

FIG. 1 schematically depicts a representative pipeline pre-commissioning operation 1 00 utilizing a non-chemical system for disinfecting test fluids. In this system, an irradiation system 110, such as an ultraviolet light treatment apparatus, renders fluid 120 biologically inert. The fluid 120 may be seawater, fresh water, or another fluid, and preferably comes from a readily available source, such as a river or the ocean. In one embodiment, the pipeline operation 100 comprises a lift pump 150, an irradiation system 110, filters 130, a pipeline fill pump 160, and a pipeline 140. The filters 130 may comprise any type of filtering apparatus to remove particles from the fluid 120, such as a sock type filter where the fluid 120 flows through a filtering insert that collects particles or any other suitable filter. The lift pump 150 and the pipeline fill pump 160 may be any type of pump suitable for moving the fluid 120 through the irradiation system 110, filters 130 and pipeline 140. The pipeline 140 may be constructed of carbon steel, an alloy, or any other material suitable for the pipeline pre-commissioning operation 100. The pumps 150, 160, the irradiation system 110, and the filters 130 may be containerized with other flow equipment and regulation instrumentation and mounted on a skid, thereby making the entire apparatus portable. In an embodiment, the skid mounted equipment is electrically powered and may be operated using generators in remote locations. Examples of such systems to treat the pipeline fill water are shown in U.S. Pat. App. Pub. No. 20070125718A1, published Jun. 7, 2007, incorporated by reference herein for all purposes.

As represented by the flow arrows, the lift pump 150 transports the fluid 120 through the filters 130, into line 180, and then into the irradiation system 110, where the filtered fluid is disinfected. The purpose of disinfection is to kill micro-organisms in the fluid 120. In an embodiment, the irradiation system 110 comprises an ultraviolet light apparatus, such as a UV-disinfection system available from HOH Water Technology A/S of Denmark, for example. The irradiation system 110 causes the deactivation of micro-organisms, thereby effectively disinfecting the fluid 120. In an embodiment, the filters 130 remove a significant quantity of debris and biological material from the fluid 120 upstream of the irradiation system 110, thereby enhancing the treatment process. In particular, the ultraviolet light source within the irradiation system 110 should penetrate through a filtered fluid more effectively than through a debris-laden fluid, and some removal of biological material upstream of the irradiation system 110 should enhance the efficiency of the irradiation treatment. In contrast to untreated fluids, such as water, irradiated fluids do not as readily corrode the wall of the pipeline 140. Further, as compared to using chemical biocides, disinfection by irradiation is more cost effective and also produces an environmentally safe fluid for disposal to the environment. After exiting the irradiation system 110, the irradiated and filtered fluid in line 185 is then transferred by the pipeline fill pump 160 through line 190 and into the pipeline 140 for use in pipeline operations, such as filling and testing procedures, for example. Once the pipeline operations are complete, the fluid exits the pipeline 140 through line 195 where the fluid may be disposed of to the environment 170 without harm thereto.

One of ordinary skill in the art will readily appreciate that the representative pipeline operation 100 of FIG. 1 may be performed offshore or onshore, and may include different components than the ones shown in FIG. 1. The pipeline operation 100 may involve pre-commissioning the pipeline 140, such as during installation and testing, or post-commissioning operations, such as a repair or replacement procedure.

The system of FIG. 1 provides disinfected fluids for testing the pipeline 140 and avoids the need to treat the fluids to remove biocides before releasing the fluids to the environment 170. However, since the fluids 120 do not contain biocides, they do not disinfect the pipeline 140. Biological materials which may be present in the pipeline 140 would remain active and may cause corrosion when the pipeline 140 is used to transport products.

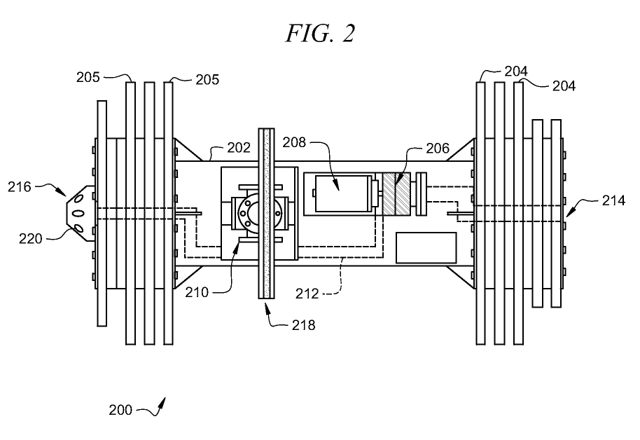

FIG. 2 illustrates an embodiment of an apparatus 200 for disinfecting the inner surface of the pipeline 140. The apparatus 200 may be referred to as a pig (or scraper), as that term is used in the pipeline art. A pipeline pig is generally a device adapted to be inserted in a pipeline and moved through the pipeline to perform various functions. For example, pigs have been used to clean and/or inspect the inner surfaces of pipelines. Pigs may be moved through pipelines by flowing fluid, such as the fill or test fluid 120, through a pipeline.

The pig 200 may have a generally cylindrical housing 202, although the housing 202 may have a square cross section or other shape. Other elements are carried in and on the housing 202. Carried on an upstream or inlet end of the housing 202 may be a one or more seal elements 204, which in this embodiment comprises a set of seal disks 204. Carried on a downstream or outlet end of the housing 202 may be one or more seal elements 205 which in this embodiment comprises a set of seal disks 205. The disks 204, 205 may be essentially circular having an outer circumference which forms a close, or preferably interference, fit with the inner surface of pipeline 140. In an embodiment, the disks 204, 205 may be formed of polyurethane, but may also be made of nylon, Delrin, Teflon, or an elastomeric material, e.g. rubber. In an embodiment, the disks 204, 205 may be formed in the shape of a dish giving a higher sealing area with the internal pipe wall. The disks 204, 205 are preferably flexible and compressible, so that they may form an essentially fluid tight seal with the inner surface of pipeline 140, but will flex so that the pig 200 may be moved through the pipeline 140 without excessive frictional resistance. The disks 204, 205 may also desirably provide a cleaning function, i.e. mechanically remove contaminants from the inner surface of the pipeline 140, as the pig 200 moves through the pipeline. The number of seal disks 204, 205 per set may be selected to achieve a desired amount of fluid tightness with the inner surface of pipeline 140. The seal elements 204, 205 may have any other shape which would restrict the flow of fluids between the pig 200 and the pipeline 140.

Carried within the housing 202 are three functional components operably coupled together; a turbine 206, a power module 208, and an irradiation source 210. A flow path 212 extends through the housing 202 and is illustrated by dashed lines. The flow path 212 extends from a fluid inlet 214 on an up stream end of the pig 200 to a fluid outlet 216 on a downstream end of the pig 200. The flow path 212 extends through the turbine 206 to direct fluid flow across turbine blades in a conventional manner. The turbine may be adapted to be driven by liquids, e.g. water or oil, or a gas, e.g. natural gas, methane, air, or nitrogen or any other fluid which may be transported through or injected into the pipeline 140. While a turbine 206 is used in this embodiment, any fluid driven or fluid powered motor, for example a piston motor, hydraulic motor, etc., may be used if desired.

A mechanical output of the turbine 206 is coupled to the power module 208 which may include a conventional rotating electrical generator. The power module 208 may also desirably include power conditioning circuitry to control voltage and current provided to the irradiation source 210. An electrical output of the power module 208 is coupled to the irradiation source 210. The irradiation source 210 may be an ultraviolet, UV, lamp like the one described above with reference to FIG. 1. In an embodiment, the UV radiation source 210 provides UV radiation at a wavelength of between about 260 nm and about 265 nm, or any other wavelength which is effective to kill or otherwise render biological material inactive.

Since the pig housing 202 needs to have substantial strength to withstand forces normally encountered in oilfield operations, it may be made of metal, such as steel or aluminum, but could be made of structural plastics. Such structurally strong materials are not normally transparent to UV radiation. Conventional UV lamps may be formed from hollow cylinders of quartz tubing. While such lamp shape fits within the housing 202, the housing 202 may have a limited number of transparent “windows” through which the radiation may be passed to irradiate the pipeline 140 without affecting the strength of the housing 202.

In an embodiment, an radiation distribution system 218 is carried on the housing 202, to receive UV radiation output from the UV source 210 and emit the UV radiation in a complete 360 degree pattern about the pig 200 so that the entire inner circumference of the pipeline 140 may be irradiated. The radiation distribution system 218 may include lenses, reflectors, optical fibers, etc. to receive the UV radiation from the UV source 210 and to direct the UV radiation to the inner surface of the pipeline over its entire inner circumference. The radiation system 218 desirably extends radially from the housing 202 to reduce the distance the UV radiation travels through fluids in the pipeline 140 before reaching the inner surface of pipeline 140.

In an alternate embodiment, one or more straight or curved UV lamps may be carried within the radiation distribution system 218, but outside the housing 202, to provide a radially uniform UV radiation pattern on the inner surface of the pipeline 140.

One use of the pig 200 is for disinfecting the pipeline 140 during the pre-commissioning process described above. Pre-commissioning includes filling the pipeline 140 with disinfected water and increasing the pressure in the pipeline 140, e.g. by means of pump 160, to determine if there are any leaks. The pig 200 may be flowed through the pipeline 140 during or ahead of the pipeline filling process, during the process of emptying the test fluid from the pipeline 140, or at any other appropriate time in the pre-commissioning or commissioning process. The pig 200 may be inserted into pipeline 140 using a conventional pig launcher located in the line 190 from pump 160 or in the inlet end of pipeline 140 where it connects to the line 190. When the pig 200 enters the pipeline 140, the disks 204, 205 preferable form a friction fit within the pipeline and essentially prevent or restrict flow of the fluid 120 between the pig 200 and the pipeline 140. As a result, fluid pumped from line 190 flows through the flowpath 212 through the pig 200. The fluid 120 therefore flows through the turbine 206, and thereby powers the power module 208. When the power module 208 generates electrical power, the UV source operates to produce UV radiation which is distributed through the radiation distribution system 218 and directed at the inner surface of the pipeline 140.

The fluid outlet 216 preferably has fluid vents 220, sized to provide a preselected fluid pressure drop across the pig 200 when at least enough fluid is flowing through the turbine 206 to produce enough power to operate the UV source 210. The number and size of disks 204, 205 may be selected to provide sufficient friction to resist movement of the pig through pipeline at the preselected pressure drop across pig 200. At start up of the disinfection process, it may be desirable to operate the pump 160 at a flow rate which produces the preselected pressure drop for a period of time to allow the UV source 210 to come up to full radiation output. Then the flow rate through the pump 160 may be increased which will increase the pressure drop across the pig 200 and overcome frictional forces between the disks 204, 205 and the inner surface of the pipeline 140. At the higher flow rate, the pig will move through the pipeline 140. It may be desirable to move the pig at a rate of about one to three feet per second to provide sufficient irradiation of the inner surface of the pipeline 140.

In the above embodiment, the sizing of vents 220 and disks 204, 205 are used to maintain sufficient pressure drop across the pig 200 to power the UV source 210 and to also regulate the speed of movement of the pig 200 through the pipeline 140. In an alternative embodiment, pressure controlled friction blocks may be used, alone or in combination with disk/vent sizing, to control the force needed to move the pig 200 through the pipeline. For example, friction blocks may be spring loaded to press the friction blocks against the pipeline 140 inner surface to resist movement of the pig 200. A piston powered by pressure drop across the pig 200 may be used to retract the friction blocks at a preselected pressure drop. Such an active system would compensate for wear of friction elements as the pig travels through a pipeline.

In an alternate embodiment, two or more pigs 200 may be passed through the pipeline 140 at the same time to ensure effective irradiation of the pipeline 140. While the pressure of pump 160 may need to be increased to drive multiple pigs 200 at the same time, the fluid flow rate may be the same.

The pig 200 may also be operated in pipeline 140 after it has been put into operation. Pipelines used to transport gas are subject to corrosion caused by hydrogen sulfide which reacts with metal to form iron corrosion products, e.g. iron sulfide, which is commonly called “black powder”. In some cases, the hydrogen sulfide is generated by sulfate reducing bacteria. The bacteria may be present on the inner surface of the pipeline and react with sulfates carried in the gas. It may be desirable to routinely pass a pig 200 through gas pipelines to disinfect the pipeline and inactivate any bacteria. As noted above, the turbine 206 may be adapted to be operated by gas as well as liquid. The size of the vents 220 may also be selected to produce an appropriate pressure drop across the pig 200 to move the pig through the pipeline 140 by the flow of gas. When disinfecting gas pipelines, it may be desirable to use known processes to remove black powder from the pipeline before disinfecting the pipeline with the pig 200. For example, a scraper pig or train of pigs may be passed through the pipeline 140 ahead of the pig 200.

The pig 200 may also be useful for disinfecting pipelines used to transport liquid hydrocarbons. Finished products, such as gasoline, jet fuel, liquid propane, etc., may be essentially transparent to UV radiation. For such finished products, the pig may be powered and driven through a pipeline by the finished product.

Opaque products, for example crude oil, may not be transparent to UV radiation and may interfere with the operation of the pig 200, by reducing the amount of radiation that reaches the inner surface of the pipeline. However, a spacer or volume of a fluid which is substantially transparent to the radiation may be inserted into a pipeline transporting opaque products (e.g., a crude oil pipeline) to permit operation of the pig 200. For example, a spacer of transparent fluid, e.g. diesel, gasoline, or disinfected water from the system of FIG. 1, may be placed into a crude oil pipeline between two conventional pigs. The conventional pigs may prevent mixing of the clear liquid with the crude oil, and may also include conventional cleaning elements such as brushes or scrapers or imaging systems for inspecting the pipeline 140. A pig 200 may be inserted behind the first conventional pig. As the spacer moves through the pipeline 140, the pig 200 would move at a slower rate, since a portion of the flowing fluid moves through the flow path 212. Thus, the pig 200 may be initially positioned near a lead pig, and then slowly move backwards (relative to the conventional pigs) within the slug toward an end pig as the pig train (e.g., lead pig, pig 200, and end pig) travels the length of the pipeline 140. The length of the transparent spacer may be selected relative to the length of pipeline 140 to be treated so that the second conventional pig (i.e., end pig) does not catch up to the pig 200 during the treating process.

Alternatively, it may be possible to fill the space between the set of disks 204 and the set of disks 205 with transparent fluid, e.g. condensate or kerosene, and drive the pig 200 with an opaque fluid such as crude oil. In this embodiment, it may be desirable to increase the number of disks 204, 205 to insure that crude oil does not flow past the disks 204, 205 and into the space around the UV distribution system 218. In an embodiment, the transparent fluid may be a transparent gel which resists flow past the disks 204, 205.

While various embodiments of hydrocarbon industry applications utilizing irradiation to disinfect a pipeline have been shown and described herein, modifications may be made by one skilled in the art without departing from the spirit and the teachings of the invention. The embodiments described herein are representative only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of any reference in the Background section is not an admission that it is prior art to the present invention, especially any reference that may have a publication date after the priority date of this application. The disclosures of all patents, patent applications, and publications cited herein are hereby incorporated by reference, to the extent that they provide representative, procedural or other details supplementary to those set forth herein.

What we claim as our invention is:

1. Apparatus for disinfecting an inner surface of a pipeline, comprising:
a housing having an upstream end and a downstream end,
at least one seal element carried on the housing, the at least one seal element sized to resist flow of fluid between the housing and the inner surface of a pipeline,
a fluid flow path extending through the housing between the upstream end and the downstream end of the housing,
a fluid powered motor coupled to the flow path,
a power generator coupled to and powered by the motor and having a power output, and
a radiation source having an input coupled to the generator power output, and having a radiation output directing radiation away from the housing.

2. The apparatus of claim 1, wherein the radiation source is an ultraviolet radiation source.

3. The apparatus of claim 2, wherein the ultraviolet radiation source provides an output of ultraviolet radiation at a wavelength of from about 260 nm to about 265 nm.

4. The apparatus of claim 1 wherein the radiation source is carried within the housing, further comprising a radiation distribution system carried on the housing, receiving radiation from the radiation source and distributing the radiation in an essentially 360 degree radial pattern around the housing.

5. The apparatus of claim 1 wherein the fluid driven motor comprises a turbine.

6. The apparatus of claim 1 wherein:
the at least one seal element comprises at least one upstream seal disk carried on the upstream end of the housing and at least one downstream seal disk carried on the downstream end of the housing, and
the radiation source output is positioned between the at least one upstream seal disk and the at least one downstream seal disk.

7. A method for disinfecting the inner surface of a pipeline, comprising:
providing a first pipeline pig comprising a fluid powered power generator having a power output, and a radiation source having an input coupled to the generator power output, and having a radiation output directing radiation away from the housing,
inserting the first pipeline pig in a pipeline,
flowing a first fluid through the pipeline at a flow rate selected to power the fluid powered power generator and selected to move the pipeline pig through the pipeline and thereby exposing the inner surface of the pipeline to radiation.

8. The method of claim 7, wherein the radiation source produces ultraviolet radiation and the first fluid is substantially transparent to ultraviolet radiation.

9. The method of claim 7, further comprising disinfecting the first fluid before flowing the first fluid through the pipeline.

10. The method of claim 9 further comprising exposing the fluid to ultraviolet radiation before flowing the first fluid through the pipeline.

11. The method of claim 7, wherein the first fluid is not transparent to the radiation, further comprising:
filling a portion of the pipeline with a spacer comprising a second fluid which is transparent to the radiation, the first pipeline pig contained within the spacer.

12. The method of claim 11, further comprising inserting a second pig in the pipeline in front of the first pipeline pig and between the first fluid and the second fluid.

13. The method of claim 12, further comprising inserting a third pig in the pipeline behind the first pipeline pig and between the first fluid and the second fluid.

14. The method of claim 7, wherein:
the first pipeline pig comprises a housing having an upstream end and a downstream end, at least one upstream seal element carried on the upstream end of the housing, and at least one downstream seal element carried on the downstream end of the housing, the seal elements sized to form a substantially fluid tight seal between the housing and the inner surface of a pipeline, and
the radiation source output is positioned between the at least one upstream seal element and the at least one downstream seal element, further comprising;
filling space between the at least one upstream seal element and the at least one downstream seal element with a second fluid which is substantially transparent to the radiation.

15. The method of claim 7 wherein the pipeline is a hydrocarbon pipeline.

16. A pipeline pig, comprising:
a fluid powered power generator having a power output, and
a radiation source having an input coupled to the generator power output, and having a radiation output directing radiation away from the pipeline pig.

17. The pipeline pig of claim 16, further comprising a radiation distributor receiving radiation from the radiation source and distributing the radiation is an essentially 360 degree radial pattern around the pipeline pig.

18. The pipeline pig of claim 16, wherein the radiation source provides an output of ultraviolet radiation at a wavelength selected to kill or inactivate biological material.

19. The pipeline pig of claim 18 wherein the radiation source provides an output of ultraviolet radiation at a wavelength of from about 260 nm to about 265 nm.

20. A method of pre-commissioning a pipeline comprising irradiating the interior surface of the pipeline prior to, during, or after pressure testing the pipeline, wherein the irradiating kills or inactivates biological material along substantially the entire interior surface of the pipeline.

21. The method of claim 20 wherein the pressure testing further comprises filling the pipeline with irradiated water.

22. The method of claim 21 wherein the interior surface is irradiated by moving a pipeline pig having an irradiation source through the pig, wherein the pipeline pig is moved, the irradiation source is powered, or both, via flow of the irradiated water through the pipeline.

23. A method of reducing corrosion in a pipeline comprising irradiating the interior surface of the pipeline to kill or inactivate biological material along substantially the entire interior surface of the pipeline.

24. The method of claim 23 wherein the pipeline is a hydrocarbon gas pipeline and wherein the biological material reacts with components of the gas in the pipeline to generate iron corrosion products.
